(19) United States
(12) Patent Application Publication
(10) Pub. No.: US 2022/0040142 A1
(43) Pub. Date: Feb. 10, 2022

(54) PHARMACEUTICAL APPLICATION FOR THE INHIBITION OF NOVEL CORONAVIRUSES BY MYRICETIN

(71) Applicant: Shanghai Spark Pharmaceutical Co., Ltd., Shanghai (CN)

(72) Inventors: Kunyuan SONG, Shanghai (CN); Weiwei CHEN, Shanghai (CN)

(21) Appl. No.: 17/287,417

(22) PCT Filed: Jun. 18, 2020
(86) PCT No.: PCT/CN2020/096824

§ 371 Date: Apr. 21, 2021

(30) Foreign Application Priority Data
Jun. 3, 2020 (CN) 202010493810.0

(51) Int. Cl. A61K 31/352; A61P 31/14

(52) U.S. Cl. CPC A61K 31/352; A61P 31/14

(57) ABSTRACT

The present disclosure provides a pharmaceutical application for the inhibition of novel coronaviruses by myricetin, which was shown to bind the 2019-nCoV-M^{pro} protease with a significant effect against the novel coronavirus (2019-nCoV) with an IC50 value of 0.582±0.0912 μM for the target M^{pro}, and was able to inhibit the 2019-nCoV virus.

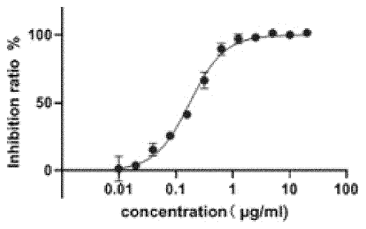

### BACKGROUND OF THE INVENTION

### Technical Field of the Invention

The present disclosure belongs to the field of pharmaceutical technology, and specifically relates to a pharmaceutical application of myricetin to inhibit novel coronavirus.

### Background

New coronary pneumonia has broken out worldwide since 2020 and may coexist with humans for a long time. There is currently no approved specific medicine available.

3-chymotrypsin-like protease, the main protease (M^{pro}, also known as 3CLpro), encoded by ORF1 (localized at nsp5), located in the central region of the replicase gene, is a key protein in the replication of novel coronavirus RNA. 3-chymotrypsin-like protease, the main protease (M^{pro}, also known as 3CLpro), encoded by ORF1 (localized at nsp5), located in the central region of the replicase gene, is a key protein in the replication of novel coronavirus RNA. The replicase polypeptides need to be further sheared into multiple proteins (e.g. RdRp, helicase, etc.), which are further assembled into the replication transcription machinery required for the virus to initiate replication of its own genetic material. M^{pro} has at least 11 cleavage sites on the replicase polypeptide, and only when these sites are cleaved properly on the replicase polypeptide is the replication transcription machinery assembled to initiate viral replication. Given that the M^{pro} protease is very important in the virus replication process, and there is no similar protein in the human body, the main protease M^{pro} has become a potential key drug target against the new coronavirus.

### SUMMARY OF INVENTION

The purpose of the present disclosure is to provide a medicine capable of inhibiting the novel coronavirus (2019-nCoV): myricetin. The present disclosure provides an application for the inhibition of novel coronaviruses by myricetin.

Furthermore, according to the application provided by the present disclosure, it may also have the feature that myricetin can bind to 2019-nCoV-M^{pro} protease, thereby inhibiting 2019-nCoV virus.

### BRIEF DESCRIPTION OF THE DRAWINGS

The FIGURE is a graphical representation of the calculated IC50 values of myricetin against M^{pro}, the target of novel coronavirus (2019-nCoV), in the experimental example of the present disclosure.

The present disclosure provides the following advantages:

The myricetin involved in the present disclosure bind the 2019-nCoV-M^{pro} protease with an IC50 value of 0.582±0.0912 μM against M^{pro}, the target of the novel coronavirus (2019-nCoV), and are significantly effective in inhibiting the 2019-nCoV virus.

### DETAILED DESCRIPTION OF THE INVENTION

In order to make the technical means, creative features, achieved purpose and effects realized by the present disclosure easy to understand, the following is a specific description of the pharmaceutical application of the myricetin or the inhibition of novel coronaviruses by myricetin. in combination with the embodiments of the present disclosure.

Myricetin: English name: Myricetin; Chinese chemical name: 3,5,7-trihydroxy-2-(3,4,5-trihydroxyphenyl)-4H-1-benzofuran-4-one; English chemical name: 3,3′,4′,5,5′,7-Hexahydroxyflavone; CAS number: 529-44-2; molecular formula: C₁₅H₁₀O₈; molecular weight: 318.24; Chemical Formula:

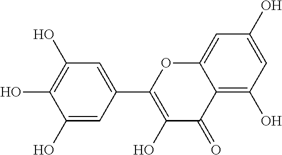 [SMILES: O=c1c(O)c(-c2cc(O)c(O)c(O)c2)oc2cc(O)cc(O)c12]

Myricetin has the effect of inhibiting the new coronavirus (2019-nCoV).

### Experimental Example

This example is the detection of M^{pro} protease activity inhibition of the targeted 2019-nCoV virus.

The fluorescence resonance energy transfer method was used to evaluate and determine the inhibitory activity of myricetin on 2019-nCoV-M^{pro} protease.

Detection method: The volume of the entire enzymatic reaction system is 120 μL, the final concentration of protease is 30 nM, and the final concentration of substrate is 20 μM. The buffer of the reaction system includes 50 mM Tris pH 7.3, 1 mM EDTA. 2019-nCoV-Mpro protease and different concentrations of myricetin were added in a 96-well plate and incubated at 30° C. for 10 min, and the substrate was added and quickly placed in an enzyme marker for counting. The excitation light and the emission light were 340 nm and 405 nm, respectively. The test time was 10 min, and the fluorescence value was read every 30 seconds. The final result was the first 2 min readings to fit the reaction rate, and compared with the control group (DMSO) to calculate the inhibition rate. Graghpad-prism 5.0 was used to make a graph, as in the FIGURE, to calculate the IC50 values for the corresponding time points of myricetin.

The test results are shown in Table 1.

TABLE 1
results of the test of myricetin-targeted
2019-nCoV-Mpro protease activity.

| Samples | IC50 (μg/ml) | IC50 (μM) |
|---|---|---|
| Myricetin | 0.185 ± 0.029 μg/ml | 0.582 ± 0.0912 μM |

From Table 1, it can be seen that the IC50 value of myricetin against M^{pro}, the target of novel coronavirus (2019-nCoV), was 0.582±0.0912 μM, which was significantly effective.

The pharmaceutical application of myricetin for inhibition of novel coronaviruses covered by the present disclosure is not limited to the scope of specific embodiments. The above is only a basic description of the present disclosure, and any equivalent transformation based on the technical solutions of the present disclosure, shall fall within the scope of protection of the present disclosure.

1. A application for the inhibition of novel coronaviruses by myricetin.

2. The application according to claim 1, wherein the myricetin bind to 2019-nCoV-M^{pro} protease, thereby inhibiting 2019-nCoV virus.
